(19) United States
(12) Patent Application Publication
(10) Pub. No.: US 2018/0090231 A1
(43) Pub. Date: Mar. 29, 2018

(54) Context-Aware Careflow Engine, Platform, Device, System, Method, and Computer-Readable Medium

(71) Applicant: Fio Corporation, Toronto (CA)

(72) Inventors: Adam Liederman, Toronto (CA); Bayan Qandil, Georgetown (CA); Ian Fine, Thornhill (CA)

(21) Appl. No.: 15/567,041

(22) PCT Filed: Apr. 15, 2016
(86) PCT No.: PCT/CA2016/000111

§ 371 Date: Oct. 16, 2017

Related U.S. Application Data
(60) Provisional application No. 62/148,468, filed on Apr. 16, 2015.

(51) Int. Cl. G16H 80/00; G16H 10/60; G16H 20/00

(52) U.S. Cl. CPC G16H 80/00; G16H 20/00; G16H 10/60

(57) ABSTRACT

The present invention is directed to a system for providing a careflow with context or context-awareness for in-process protocols directed at the patient with a goal to providing optimal patient care. The contextual awareness is based on information which may be internal or external to the patient, with such information comprising both medical and non-medical information.

### PRIORITY

The present application claims the benefit of the earlier filed U.S. Patent Provisional Application No. 62/148,468 filed on Apr. 16, 2015.

### FIELD OF THE INVENTION

The present invention relates generally to patient-centered and/or context-aware careflow engine, platform, device, system, method, computer-readable medium, and/or cooperating environment.

### BACKGROUND OF THE INVENTION

Improving patient care and tracking clinical outcomes is a priority worldwide. So called “evidence-based” medicine (e.g. the conscientious and explicit use of current best evidence in making decisions about the care of individual patients) has been widely promoted as a way of improving and tracking such patient care and clinical outcomes. In current health care systems, however, scientific knowledge about best patient care may not be applied systematically or expeditiously to clinical practice and clinical guidelines. The resulting variability in such clinical practice and clinical guidelines in which there may be a strong scientific evidence and a high degree of expert consensus about best practice indicates that current dissemination efforts fail to reach many clinicians and patients, and that there are insufficient tools and incentives to promote the rapid adoption of such clinical practice and clinical guidelines.

Clinical guidelines and practices describe the activities of a medical team in a comprehensive manner for the purpose of defining best practice for patient care management. Focusing on the behavioural aspects of medical work with regard to supporting applicable clinical practice and guidelines may be referred to as “careflow”. Careflow systems and methods implement workflow concepts in the clinical domain. Such careflow involves the co-ordinated execution of multiple tasks which may be performed by different actors (i.e. doctors, technicians, patients, etc.). These tasks can be manual, or automated, either created specifically for any given medical context, each application being developed, or other care specific parameters.

The activities of a care providers' medical team should be coordinated within a process properly designed on the basis of available best practice medical knowledge. This may require a rethinking of the management of care processes within health care organizations. The current workflow technology seems to offer the most convenient solution to build such cooperative systems. However, some of its present weaknesses still require an intense research effort to find solutions allowing its exploitation in real medical practice.

To deliver optimal care, healthcare providers require (a) comprehensive information on the patient's condition and medical context; (b) time to study that information in order to make thoughtful conclusions; and (c) deep knowledge of standardized evidence-based protocols for making clinical management decisions. In practice, however, healthcare providers and users (medical practitioners, technicians, patients, etc.) may (a) have access only to partial information (e.g., only information gathered firsthand if no historical record may be available, or only partial information if a patient's history may be spread across multiple dis-integrated electronic record systems); (b) are under time pressure to move onto subsequent patients; and (c) are unfamiliar with the latest protocols and/or fail to adhere to the oftentimes complex protocols that guide single clinical encounters let alone those that span multiple healthcare functions, facilities and time.

All of these problems become more extreme as care moves outside of hospitals and toward decentralized facilities where there may be even less access to up-to-date records (e.g. electronic records), clinical decision support tools, and peers. Limitation of low-resource settings in highly rural or developing world countries further compounds the challenges.

Furthermore, protocols are even more effective when supplemented by context-specific information to provide in-process guidance. Such context-specific information (e.g. family and personal history, travel history, previous medical history, etc.) from various sources can be vital during patient care interactions. Providing this information at the appropriate time in a manner than can enhance patient care may be vital.

As a result, there may be a need for, or it may be desirable to provide, one or more patient-centered and/or context-aware careflow engines, platforms, devices, systems, methods, computer-readable media, and/or cooperating environments.

It may be an object of the present invention to obviate or mitigate one or more disadvantages and/or shortcomings associated with the prior art, to meet or provide for one or more needs and/or advantages, and/or to achieve one or more objects of the invention—one or more of which may preferably be readily appreciable by and/or suggested to those skilled in the art in view of the teachings and/or disclosures hereof.

### SUMMARY OF THE INVENTION

The present disclosure provides a method, computer-readable medium and system for coordinating healthcare services. More specifically, embodiments of the present invention are directed to systems for context-awareness for in-process patient care management comprising: (a) a first database in communication with a network and configured to store therein a shared patient record of a patient and at least one patient careflow for treatment of a condition of the patient; (b) a careflow engine in communication with the network and configured to receive the shared patient record and the at least one patient careflow therefrom and to analyze the careflow in the context of the shared patient record in order to create a clinical careflow; (c) a notification engine in communication with the network and configured to receive the clinical careflow from the careflow engine and to communicate the clinical careflow to a health care provider who is co-located with the patient during or before the time of treatment of the patient; and (d) a network ready device in communication with the network and configured to display the clinical careflow to the health care provider through a health care provider interface for the health care provider to guide the treatment of the condition of the patient with the clinical careflow.

In another embodiment of the present invention, there is provided a method for providing context-awareness during in-process patient care comprising: (a) storing an electronic record of a patient and at least one patient careflow for treatment of a condition of the patient in a first database communicating with a network; (b) transmitting the electronic record of a patient and the at least one patient careflow through the network to a careflow engine communicating with the network, the careflow engine analyzing the careflow in the context of the electronic record of a patient and creating a clinical careflow; (c) transmitting the clinical careflow through the network to a notification engine communicating with the network, the notification engine transmitting the clinical careflow to a network ready device of a health care provider who is in-process with the patient during or before the time of treatment of the patient; and (d) displaying the clinical careflow to the health care provider through a health care provider interface in the network ready device of the health care provider guiding the treatment of the condition of the patient.

In yet another embodiment of the present invention, there is provided a non-transitory computer readable medium encoded with executable instructions for providing context-awareness during in-process patient care, the executable instructions comprising code for: (a) storing an electronic record of a patient and at least one patient careflow for treatment of a condition of the patient in a first database communicating with a network; (b) transmitting the electronic record of a patient and the at least one patient careflow through the network to a careflow engine communicating with the network, the careflow engine analyzing the careflow in the context of the electronic record of a patient and creating a clinical careflow; (c) transmitting the clinical careflow through the network to a notification engine communicating with the network, the notification engine transmitting the clinical careflow to a network ready device of a health care provider who is in-process with the patient during or before the time of treatment of the patient; and (d) displaying the clinical careflow to the health care provider through a health care provider interface in the network ready device of the health care provider guiding the treatment of the condition of the patient.

In a preferred embodiment of the present invention, the above noted system, method or computer readable medium further provides that the patient careflow further comprises treatment workflows.

In yet a further preferred embodiment of the present invention, the above noted system, method or computer readable medium further provides that the shared patient record comprises an electronic health record.

In yet a further preferred embodiment of the present invention, the above noted system, method or computer readable medium further provides that the electronic health record further comprises an electronic medical record.

In yet a further preferred embodiment of the present invention, the above noted system, method or computer readable medium further provides that the shared patient record further comprises medical and non-medical patient specific information.

In yet a further preferred embodiment of the present invention, the above noted system, method or computer readable medium further provides that the medical and non-medical patient specific information is selected from the group consisting of healthcare providers having encounters with the patient, internal patient factors and external patient factors.

In yet a further preferred embodiment of the present invention, the above noted system, method or computer readable medium further provides that the internal patient factors comprise the patient's medical history.

In yet a further preferred embodiment of the present invention, the above noted system, method or computer readable medium further provides that the external patient factors comprise geographic information, social media information, non-medical personal information.

In yet a further preferred embodiment of the present invention, the above noted system further provides that the treatment workflows comprise clinical practice protocols or guidelines.

In yet a further preferred embodiment of the present invention, the above noted system, method or computer readable medium further provides that the careflow engine evaluates the patient careflow and the shared patient record to determine the next treatment workflow in the treatment of the condition of the patient.

In yet a further preferred embodiment of the present invention, the above noted system, method or computer readable medium further provides that the careflow engine evaluates the patient careflow and the shared patient record to determine if deviations from the patient careflow have occurred.

In yet a further preferred embodiment of the present invention, the above noted system, method or computer readable medium further provides that the careflow engine is configured to receive data from a second database, where the second database is selected from a group consisting of proprietary databases, epidemiologic databases, medical records databases, UN and major/international healthcare institution databases, healthcare and emergency infrastructure databases, education and economic databases, news databases, demographic databases, communication and military infrastructure databases, and weather, travel, topographic databases.

In yet a further preferred embodiment of the present invention, the above noted system, method or computer readable medium further provides that the notification engine is configure to anonymize the clinical careflow and to communicate the anonymized clinical careflow to a governmental or non-governmental agency responsible for the patient careflow.

In yet a further preferred embodiment of the present invention, the above noted system, method or computer readable medium further provides that the healthcare provider is selected from the group consisting of a primary care physician, a nurse practitioner, a clinic, a hospital, a physician's assistant, a therapist, a specialist, an insurance carrier, a healthcare payer, a pharmacist, an accountable care organization, and a hospice provider.

In yet a further preferred embodiment of the present invention, the above noted system, method or computer readable medium further provides that the first database, the second database, the careflow engine, the notification engine and the network ready device are configured to communicate securely, and preferably in-process, using at least one of email, short message service (SMS), secure mobile messaging, web hooks, system dashboards, interactive voice response, etc.

### BRIEF DESCRIPTION OF THE DRAWINGS

The novel features which are believed to be characteristic of the system, device and methods according to the present invention, as to their structure, organization, use, and method of operation, together with further objectives and advantages thereof, may be better understood from the following drawings in which presently preferred embodiments of the invention may now be illustrated by way of example. It is expressly understood, however, that the drawings are for the purpose of illustration and description only, and are not intended as a definition of the limits of the invention. In the accompanying drawings:

FIG. 1 is a schematic diagram of an embodiments of the present invention;

FIG. 2 is a schematic diagram of another embodiment of the present invention;

FIG. 3 is a schematic diagram of yet another embodiment of the present invention;

FIG. 4 is a flowchart of yet another embodiment of the present invention;

FIG. 5 is a schematic diagram of yet another embodiment of the present invention;

### DETAILED DESCRIPTION OF THE PREFERRED EMBODIMENTS

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order to more clearly depict certain embodiments and features of the invention.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

The present disclosure may be now described in terms of an exemplary system in which the present disclosure, in various embodiments, would be implemented. This may be for convenience only and may be not intended to limit the application of the present disclosure. It may be apparent to one skilled in the relevant art(s) how to implement the present disclosure in alternative embodiments.

In this disclosure, a number of terms and abbreviations may be used. The following definitions and descriptions of such terms and abbreviations are provided in greater detail.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

As used herein, a person skilled in the relevant art may generally understand the term “healthcare provider” to refer to the group of healthcare workers (“HCW”) and others who are involved, directly or indirectly in patient care, encounters and medical events, including but not limited to, primary care physicians, nurse practitioners, clinics, hospitals, physician's assistants, therapists, medical specialists, medical technicians, insurance carriers, healthcare payors, pharmacists, an accountable care organization, hospice providers, etc.

It should also be appreciated that the present invention can be implemented in numerous ways, including as a process, method, an apparatus, a system, a device, a method, or a computer readable medium such as a computer readable storage medium or a computer network wherein program instructions are sent over a network (e.g. optical or electronic communication links). In this specification, these implementations, or any other form that the invention may take, may be referred to as processes. In general, the order of the steps of the disclosed processes may be altered within the scope of the invention.

Preferred embodiments of the present invention can be implemented in numerous configurations depending on implementation choices based upon the principles described herein. Various specific aspects are disclosed, which are illustrative embodiments not to be construed as limiting the scope of the disclosure. One aspect of the disclosure is for data driven method, computer program product, apparatus, and system for context awareness and interoperability in the context of patient care. Although the present specification describes components and functions implemented in the embodiments with reference to standards and protocols known to a person skilled in the art, the present disclosures as well as the embodiments of the present invention are not limited to any specific standard or protocol. Each of the standards for non-mobile and mobile computing, including the Internet and other forms of computer network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same functions are considered equivalents.

As those of ordinary skill in the art would generally understand, the Internet is a global computer network which comprises a vast number of computers and computer networks which are interconnected through communication links. A person skilled in the relevant art may understand that an electronic communications network of the present invention, may include, but is not limited to, one or more of the following: a local area network, a wide area network, peer to peer communication, an intranet, or the Internet. The interconnected computers exchange information using various services, including, but not limited to, electronic mail, Gopher, web-services, application programming interface (API), File Transfer Protocol (FTP) This network allows a server computer system (a Web server) to send graphical Web pages of information to a remote client computer system. The remote client computer system can then display the Web pages via its web browser. Each Web page (or link) of the “world wide web” (“WWW”) is uniquely identifiable by a Uniform Resource Locator (URL). To view a specific Web page, a client computer system specifies the URL for that Web page in a request (e.g., a HyperText Transfer Protocol (“HTTP”) request). The request is forwarded to the Web server that supports the Web page. When the Web server receives the request, it sends the Web page to the client computer system. When the client computer system receives the Web page, it typically displays the Web page using a browser. A web browser or a browser is a special-purpose application program that effects the requesting of web pages and the displaying of web pages and the use of web-based applications. Commercially available browsers include Microsoft Internet Explorer and Firefox, Google Chrome among others. It may be understood that with embodiments of the present invention, any browser would be suitable.

A person skilled in the relevant art would generally understand that a reference to “Internet of Things (IoT)” or “IoT device” refers to networked or interconnected objects, typically, but not limited to, everyday objects, more technically purposed objects (i.e. medical devices) and devices. It is described as a self-configuring wireless network of sensors whose purpose would be to interconnect all such connected devices. The concept may be attributed to the former Auto-ID Center, founded in 1999, based at the time at the Massachusetts Institute of Technology (MIT).

Web pages are typically defined using HTML. HTML provides a standard set of tags that define how a Web page is to be displayed. When a provider indicates to the browser to display a Web page, the browser sends a request to the server computer system to transfer to the client computer system an HTML document that defines the Web page. When the requested HTML document is received by the client computer system, the browser displays the Web page as defined by the HTML document. The HTML document contains various tags that control the displaying of text, graphics, controls, and other features. The HTML document may contain URLs of other Web pages available on that server computer system or other server computer systems.

A person skilled in the relevant art may generally understand a web-based application refers to any program that is accessed over a network connection using HTTP, rather than existing within a device's memory. Web-based applications often run inside a web browser or web portal. Web-based applications also may be client-based, where a small part of the program is downloaded to a user's desktop, but processing is done over the Internet on an external server. Web-based applications may also be dedicated programs installed on an internet-ready device, such as a smart phone or tablet. A person skilled in the relevant art may understand that a web site may also act as a web portal. A web portal may be a web site that provides a variety of services to users via a collection of web sites or web based applications. A portal is most often one specially designed site or application that brings information together from diverse sources in a uniform way. Usually, each information source gets its dedicated area on the page for displaying information (a portlet); often, the user can configure which ones to display. Portals typically provide an opportunity for users to input information into a system. Variants of portals include “dashboards”. The extent to which content is displayed in a “uniform way” may depend on the intended user and the intended purpose, as well as the diversity of the content. Very often design emphasis is on a certain “metaphor” for configuring and customizing the presentation of the content and the chosen implementation framework and/or code libraries. In addition, the role of the user in an organization may determine which content can be added to the portal or deleted from the portal configuration.

A person skilled in the relevant art will generally understand that the term “engine” refers to computer program, or part of a computer program, that serves as the core foundation for a larger piece of software. The term “engine” can be used by developers when referencing a library, SDK or object, to denote an encapsulated block of functionality. As used herein, the term “module” or “component” can also refer to software engines, objects or routines that execute on the computing system. The different components, modules, engines, and services described herein may be implemented as objects or processes that execute on the computing system.

A person skilled in the relevant art would generally understand that the term “medical condition” or “condition” to refer to a condition that requires the input, treatment or review by a healthcare provider, including a disease, illness or injury; any physiologic, mental or psychological condition or disorder (e.g., orthopaedic; visual, speech or hearing impairments; cerebral palsy; epilepsy; muscular dystrophy; multiple sclerosis; cancer; coronary artery disease; diabetes; emotional or mental illness; specific learning disabilities; infectious disease; drug addiction; alcoholism; pregnancy, etc.).

In the following specification, it may be understood by a person skilled in the relevant art that the term “careflow” refers to the activities and behaviour aspects of members of a medical team (or individual members of the team) in the application and implementation of clinical guidelines, clinical practices, patient information (e.g. patient “context”), etc. in a comprehensive manner for the purpose of implementing and further defining and refining best practices for patient care management. It may be further understood by a person skilled in the relevant art that the term “workflow” refers to the specific activities that may be conducted with respect to or during the careflow. Examples of “workflow” may include, specific medical tests, procedures, questionnaires, etc. that may be necessary, advisable or preferred with a particular careflow system and method. A workflow may also be a series or set of steps that the healthcare worker must perform, preferably in sequence. These could be asking simple questions, checking basic vitals or running diagnostic tests using a medical or test device. Careflow systems and methods implement one or more workflows in the clinical domain. Such careflow systems and methods involves the co-ordinated execution of multiple tasks (e.g. workflows) which may be performed by different actors (i.e. doctors, technicians, patients, etc.). These tasks can be manual, or automated, either created specifically for any given medical context, each application being developed, or other care specific parameters. In a preferred embodiment, workflow protocols are medical condition dependent and can range from a set of questions to as many as, but not limited or restricted to, up to 30 activities; careflow protocols may provide for one or more workflows throughout the lifetime of the patient and/or the lifetime of the condition. Over a patient lifetime, the patient could be subject to multiple careflow(s). It may be generally understood that in accordance with the present invention, careflow(s) and/or workflow(s) may be dynamic and change over time depending on specific input. One patient could have careflows for a viral infection (e.g. HIV), heart disease and diabetes, simultaneously or sequentially. In addition, that patient may have traveled to a specific geographic location. As such, it would be understood that the workflows for each of the se careflows could interact and therefore need to be aware of each other so that the best medical outcome can be achieved for the patient.

A preferred aspect of the present invention may be directed to a careflow engine that provides context or context-awareness for in-process (e.g. during or adjacent to workflows or careflow) protocols about the patient in a larger informational context with a goal to providing optimal patient care. The informational context may be based on information which may be internal or external to the patient, with such information comprising both medical and non-medical information. A person skilled in the relevant art would generally understand that the external and internal factors may be both medical and non-medical related information. Geographic information, “social media” information (TWITTER™, FACEBOOK™, etc.), non-medical personal information (i.e. calendar information, travel information, etc.) and the like could be used to develop context awareness in accordance with the present invention. Prior art careflow and/or workflow systems are not context aware and are dependent on the information provided by the patient or the medical record of that patient which may be incomplete at best. An aspect of context-awareness provides for the ability for the careflow to modify the workflow and vis-versa, preferably in-process. For example, if a patient presents with a sore throat, the healthcare provider (e.g. a healthcare worker) may simple prescribe antibiotics using prior art methods. If, however, the healthcare provider knew (or the present invention made the healthcare provider aware) of the fact that the patient had been to a walk-in clinic three times previously and received the same treatment as that being proposed, the healthcare provider may decide to proceed with the another treatment protocol (e.g. different medications, send patient to a medical specialist, etc.). An aspect of the present invention is obtaining information relevant to the health care provider (e.g. medical event and/or clinically relevant information) at the relevant time, namely at, before or during the point of care (“PoC”). This timing may be generally known as or can be referred to as “in-process” (e.g. during the workflow/careflow process).

Another aspect of the present invention is the ability to drive careflow and/or workflow creation and modification. Aspects of the present invention provide the applicable decision maker with regard to the careflow/workflow protocols (e.g. a ministry of health (“MOH”), other governmental, non-governmental agency, etc.), the ability to decide how it may want to proceed with existing protocols or whether they are to be amended, modified, deleted or changed, based on a greater source of information delivery at an applicable time. The careflow engine of the present invention does not define the protocols but the embodiments of the present invention allows the delivery of relevant information so as to provide for customizable careflow and workflow protocols and how to use the relevant information in a more efficient and appropriate manner. The notifications from the present invention that are sent to the MOH or any other decision maker, to help them define more effective workflow or careflows it could also be provided to the healthcare provider to alter the decision making process for more effective patient treatment. Providing the information and the tools to disseminate the information to the end users (e.g. health care provider, including, but not limited to a health care worker) to implement the careflow(s) and/or workflow(s). The embodiments of the present invention allow the MOH or other applicable entity responsible for the workflow/careflow to amend or modify the careflow and/or workflow to allow for better outcomes for the patient.

It may be generally understood by a person skilled in the relevant art that the term “mobile device” or “portable device” refers to any portable electronic device that can be used to access a computer network such as, for example, the internet. Typically a portable electronic device comprises a display screen, at least one input/output device, a processor, memory, a power module and a tactile man-machine interface as well as other components that are common to portable electronic devices individuals or members carry with them on a daily basis. Examples of portable devices suitable for use with the present invention include, but are not limited to, smart phones, cell phones, wireless data/email devices, tablets, PDAs and MP3 players, test devices, etc.

It may be generally understood by a person skilled in the relevant art that the term “network ready device” or “internet ready device” refers to devices that are capable of connecting to and accessing a computer network, such as, for example, the Internet, including but not limited to an IoT device. A network ready device may assess the computer network through well-known methods, including, for example, a web-browser. Examples of internet-ready devices include, but are not limited to, mobile devices (including smart-phones, tablets, PDAs, etc.), gaming consoles, and smart-TVs. It may be understood by a person skilled in the relevant art that embodiment of the present invention may be expanded to include applications for use on a network ready device (e.g. cellphone). In a preferred embodiment, the network ready device version of the applicable software may have a similar look and feel as a browser version but that may be optimized to the device. It may be understood that other “smart” devices (devices that are capable of connecting to and accessing a computer network, such as, for example, the internet) such as medical or test devices, including but not limited to smart blood pressure monitors, smart glucometers, IoT devices, etc.

It may be further generally understood by a person skilled in the relevant art that the term “medical event” (“ME”) references a medical occurrence or set of occurrences for which treatment may be required. In a preferred embodiment, a medical event refers to the outcome of one or more workflows, for example, the result from a diagnostic test, suggested treatment, patient's response to treatment (e.g., clinical outcome).

It may be further generally understood by a person skilled in the relevant art that the term “downloading” refers to receiving datum or data to a local system (e.g. mobile device) from a remote system (e.g. a client) or to initiate such a datum or data transfer. Examples of a remote systems or clients from which a download might be performed include, but are not limited to, web servers, FTP servers, email servers, or other similar systems. A download can mean either any file that may be offered for downloading or that has been downloaded, or the process of receiving such a file. A person skilled in the relevant art may understand the inverse operation, namely sending of data from a local system (e.g. mobile device) to a remote system (e.g. a database) may be referred to as “uploading”. The data and/or information used according to the present invention may be updated constantly, hourly, daily, weekly, monthly, yearly, etc. depending on the type of data and/or the level of importance inherent in, and/or assigned to, each type of data. Some of the data may preferably be downloaded from the Internet, by satellite networks or other wired or wireless networks.

Elements of the present invention may be implemented with computer systems which are well known in the art. Generally speaking, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (“LAN”). Networking environments are commonplace in offices, enterprise-wide computer networks and home computer systems. A wide area network (“WAN”), such as the Internet, can also be accessed by the computer or mobile device.

It may be appreciated that the type of connections contemplated herein are exemplary and other ways of establishing a communications link between computers may be used in accordance with the present invention, including, for example, mobile devices and networks. The existence of any of various well-known protocols, such as TCP/IP, Frame Relay, Ethernet, FTP, HTTP and the like, may be presumed, and computer can be operated in a client-server configuration to permit a user to retrieve and send data to and from a web-based server. Furthermore, any of various conventional web browsers can be used to display and manipulate data in association with a web based application.

The operation of the network ready device (i.e. a mobile device) may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention can be implemented by a software program for processing data through a computer system. It may be understood by a person skilled in the relevant art that the computer system can be a personal computer, mobile device, notebook computer, server computer, mainframe, networked computer (e.g., router), workstation, and the like. In one embodiment, the computer system includes a processor coupled to a bus and memory storage coupled to the bus. The memory storage can be volatile or non-volatile (i.e. transitory or non-transitory) and can include removable storage media. The computer can also include a display, provision for data input and output, etc. as may be understood by a person skilled in the relevant art.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers or the like.

It may be generally understood that in establishing a user interface, a task bar may be preferably positioned at the top of a screen to provide a user interface. Preferably, a textual representation of a task's name is presented in this user interface, preferably as a button, and the task names may be shortened as necessary if display space of the button is constrained. The labelled button having the task's name preferably operate as a type of hyperlink, whereby the user/viewer can immediately switch to the activity, view, etc. of each of the tasks by selecting the button containing the applicable name from the task bar. In other words, the user or viewer is redirected by the application to the function represented by the task button by selecting the labelled hyperlink. Preferably, the task entry associated with the currently-displayed work unit view may be shown in a different graphical representation (e.g., using a different color, font, or highlighting). In preferred embodiments, there may be provided a display having a selectable “X” in the task bar entry for each task: if the user clicks on the “X”, then its associated task may be ended and the view of its work unit may be removed. A user interface may be web-based, application based, or a combination.

In accordance with a preferred aspect of the present invention, a person skilled in the relevant art would generally understand the term “application” or “application software” to refer to a program or group of programs designed for end users. While there are system software, typically but not limited to, lower level programs (e.g. interact with computers at a basic level), application software resides above system software and may include, but is not limited to database programs, word processors, spreadsheets, etc. Application software may be grouped along with system software or published alone. Application software may simply be referred to as an “application”.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices. As used herein, reference to the “execution”, “processing,” “interpretation” or the like of careflow(s), workflow(s), clinical guidelines, etc. may refer to advancing through logic contained in the guideline. This may be accomplished, among other methods, by running on a processor one or more computer programs representative of the careflows, workflow, clinical guidelines, etc.

According to the invention, one or more patient-centered and/or context-aware careflow engines, platforms, devices, systems, methods, computer-readable media, and/or cooperating environments may be disclosed.

The invention is contemplated for use in association with one or more cooperating environments, to afford increased functionality and/or advantageous utilities in association with same. The invention, however, is not so limited.

Certain novel features which are believed to be characteristic of the engine, platform, device, system, method, computer readable medium, and/or certain features of the engine, platform, device, system, method, computer readable medium which are novel in conjunction with the cooperating environment, according to the present invention, as to their organization, use, and/or method of operation, together with further objectives and/or advantages thereof, may be better understood from the accompanying disclosure in which presently preferred embodiments of the invention are disclosed by way of example. It is expressly understood, however, that the accompanying disclosure is for the purpose of illustration and/or description only, and is not intended as a definition of the limits of the invention.

Naturally, in view of the teachings and disclosures herein, persons having ordinary skill in the art may appreciate that alternate designs and/or embodiments of the invention may be possible (e.g., with substitution of one or more steps, algorithms, processes, features, structures, parts, components, modules, utilities, etc. for others, with alternate relations and/or configurations of steps, algorithms, processes, features, structures, parts, components, modules, utilities, etc).

Although some of the steps, algorithms, processes, features, structures, parts, components, modules, utilities, relations, configurations, etc. according to the invention are not specifically referenced in association with one another, they may be used, and/or adapted for use, in association therewith.

One or more of the disclosed steps, algorithms, processes, features, structures, parts, components, modules, utilities, relations, configurations, and the like may be implemented in and/or by the invention, on their own, and/or without reference, regard or likewise implementation of one or more of the other disclosed steps, algorithms, processes, features, structures, parts, components, modules, utilities, relations, configurations, and the like, in various permutations and combinations, as may be readily apparent to those skilled in the art, without departing from the pith, marrow, and spirit of the disclosed invention.

Embodiments of the present invention are directed to systems, methods and computer readable media that provide in-process (e.g. during the process of patient care) clinical decision support with context awareness to health workers in any setting, from hospitals to decentralized, low-resource facilities. In a preferred embodiment, the preferred aspects of the invention may comprise on or more of the following: (a) a “data store” (e.g. a database) combined with a shared health record system to make patient context information available; (b) a means to access digitally encoded clinical practice protocols or guidelines (e.g. careflows and/or workflows) so healthcare providers need not commit such clinical practice protocols or guidelines to memory; (c) a careflow engine to analyze the patient information in the context of the careflow(s) and/or workflow(s) in order to create or formulate clinical careflow(s) and/or workflow(s) to provide guidance to the healthcare provider; (d) notification engines to communicate the foregoing to healthcare providers while they are co-located with patients; (e) electronic devices (e.g., mobile phones, tablets, computers, etc.) and user interfaces for healthcare providers to interact with the preferred embodiments of the present invention, preferably through a graphic user interface (“GUI”).

An embodiment of the present invention presents relevant information to the health care provider contextually in-process at the appropriate time to support high quality care delivery. In a preferred embodiment, one or more data stores contain relevant patient data, which may include medical and non-medical information that may be internal or external to the patient. The “careflow engine” may be context-aware as it (a) evaluates the patient specific careflow(s) and relevant patient data; (b) determines the next logical encounter or workflow that must be conducted; (c) determines if deviations from the patient specific careflow have happened (such as, for example, but not limited to, missed workflows by healthcare providers or missed encounters by patients); and (d) provides in-process notifications to relevant stakeholders, such as, for example, but not limited to, healthcare providers and careflow/workflow decision makers (e.g. MOH), if necessary. With ‘data exchange’, ‘interoperability’ and ‘standards’, such context awareness may also work with or involve other software provider solutions as well allowing for improved delivery of care. The lack of the forgoing in prior art systems become more extreme as healthcare moves outside of hospitals and toward decentralized facilities (e.g. highly rural or developing world countries) where there may be even less access to up-to-date records (e.g. electronic records), clinical decision support tools, and peers.

The preferred embodiments of the present invention are directed to creating systems and methods that allow for the connection of separate technology and information databases and systems, adding a patient-centered context-aware careflow engine, working across healthcare functions, supporting multiple data systems, supplementing missing services, controlling data access, and/or providing actionable information through a single portal.

Furthermore, protocols are even more effective when supplemented by context-specific information to provide in-process (e.g. workflow and careflow) guidance. Such context-specific information (e.g. family and personal history, travel history, previous medical history, etc.) from various sources provides benefit during patient care interactions. Providing this information at the appropriate time in a manner that can also enhance patient care is an aspect of the present invention.

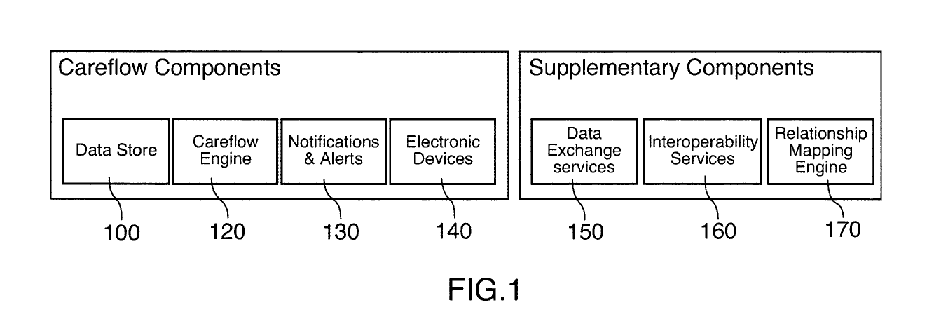

As shown in FIG. 1, there is provided the preferred components of the present invention. There is provided a data store 100, a careflow engine 120, a notification and alert engine 130 and various networked devices 140. In a further preferred embodiment, there is provided data exchange services 150, interoperability services 160 and relationship mapping engine 170. Each of these components are described in greater detail below.

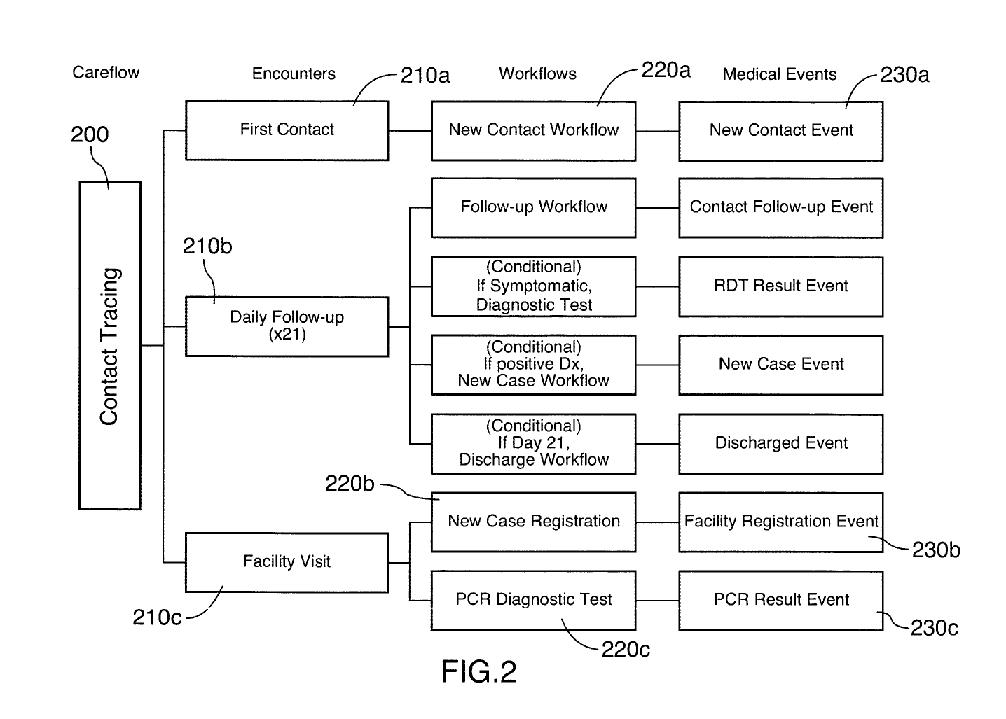

As shown in FIG. 2, there is outlined the careflow and associated workflow for a “contact tracing” careflow that may, but not necessarily, be utilized when healthcare providers are dealing with infectious disease pandemics. As shown in FIG. 2, the applicable contact tracing careflow 200 involved in the contact tracing protocol is structured and supports repetition and conditions allowing for complex scenarios to be defined. A careflow comprises one or more groups of workflows, which may be referred to as encounters 210, such as a “first contact” encounter 210a, daily follow-up encounter 210b or a facility visit encounter 210c. An encounter may, but not necessarily, be a type of interaction between a health care provider and with a patient. As shown in FIG. 2, there is also provided one or more workflow 220, which may comprise the set of steps that the healthcare provider performs. These could include asking simple questions, checking basic vitals or running diagnostic tests using IVD devices. Each workflow derives from an encounter; single or multiple workflows may arise from one encounter. For example, encounter 210a gives rise to a single workflow 220a, while encounter 210c gives rise to two workflows 220b and 220c. As shown in FIG. 2, there is also provided one or more medical event 230 which reflects the outcome of one or more workflows 220. In other words, the medical events may be seen as the results of a planned workflow; medical event 230a is a result of workflow 220a, for example. As can be seen from FIG. 2, each careflow 200 has multiple possible patient encounters (210a, 210b, 210c) and each patient encounter is dealt with by one or more workflows (220a, 220b, 220c).

Within the workflow, there are specific criteria (e.g. clinical guidelines, protocols, etc.) that may be followed. In prior art systems, each workflow is conducted without input based on other existing workflow or careflows. When a patient presents with a sore throat, for example, the patient may also be HIV positive and enrolled in an HIV careflow which may track the patient over a period of time. This HIV careflow may have certain steps or criteria to be followed over a certain period of time and by doing so. At the workflow level (see 220 in FIG. 2), if the patient is being evaluated for something, which may or may not be related to the larger careflow (i.e. HIV), such as, for example, cold, flu, infection, etc., embodiment of the present invention may pull in such required context awareness. In other words, there may be specific patient external or internal factors (e.g. a malaria or Zika virus outbreak, increase in blood pressure) that may be relevant to the workflow/careflow that is not currently being captured.

In a preferred embodiment of the present invention, the system may store one or more careflows as having expected future medical events, along with when they are expected as per the applicable protocol.

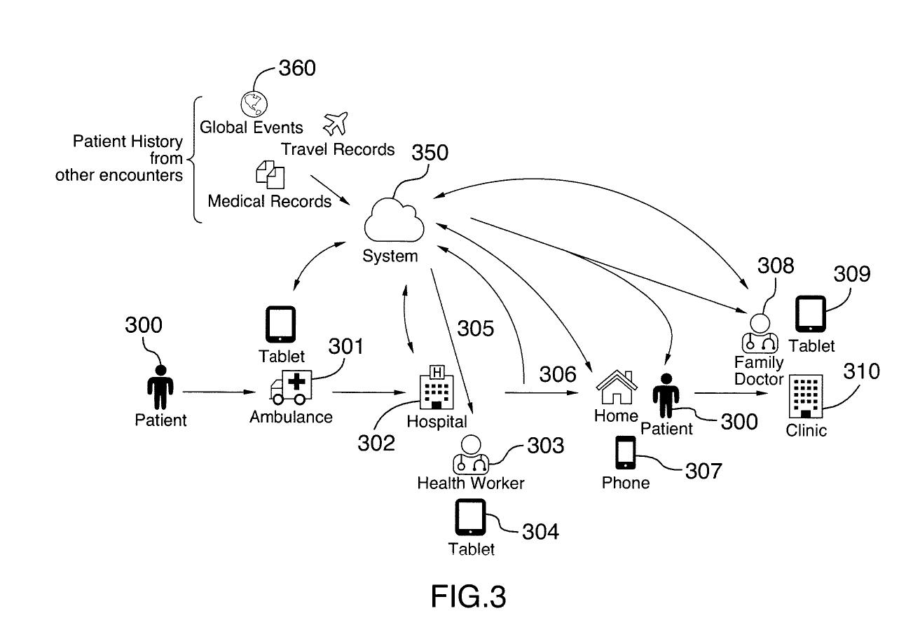

As shown in FIG. 3, there is provided an outline of a preferred embodiment of the present invention. There is provided a system 350 of the present invention which is connected to a number of data stores 360 having patient related medical and nonmedical information. Where a patient 300 presents to a healthcare provider 302 (e.g. hospital) via ambulance 301, a healthcare worker 303 looks to some type of careflow and/or workflow protocols (either preassigned or not yet assigned) to be conducted to direct the care or treatment of the patient 300. On route to hospital 302, other health care workers (e.g. paramedics) may obtain patient information (i.e. previous trauma, allergies, etc) (not shown). Paramedics may also check vitals and input into a mobile device, which may automatically push such information to the careflow engine of the present invention. Upon arrival at hospital 302, healthcare worker may have has access to patient information via a mobile device 304.

A patient-specific alert 305 may be provided to healthcare worker 303 during patient examination (i.e. patient recently traveled to area with confirmed Zika outbreak, and vitals showed symptoms consistent with Zika). At 306, in FIG. 3, patient 300 may be discharged and information related to health, medications, follow-ups needed may be recorded and all data is sent to system 350. Patient 300 follows medicine regimen, takes measurements on smart medical devices (i.e. connected BP monitor), etc.

As shown in FIG. 3, family physician 308 and/or clinic 310 can track patient progress remotely using tablet 309. Physician 308 is also presented with relevant patient-specific information. The family physician can also adjust treatment remotely and communicate with patient. If the patient 300 misses a follow-up or a critical portion of their treatment, patient 300, clinic 310 and/or family physician 308 can be alerted of the error via system 350.

Currently, during the execution of these careflow or workflow protocols, there is no contextual awareness, and as such, the patient may receive sub-optimal care. The embodiments of the present invention provide that during the execution of the careflow and/or workflow protocols, contextual awareness can be provided to provide optimal patient care. In the middle of workflow or careflow protocols (referred to as careflows or workflows), context dependent information (e.g. medical or non-medical information) are pulled in to give more relevant information to the healthcare provider or MOH for patient treatment. In a preferred embodiment, this information may be provided to the healthcare provider directly or to MOH via notifications through a notification engine.

As a further example of a preferred embodiment of the present invention, a patient who recently traveled to a geographical location where there is a particular viral outbreak (e.g. dengue fever) may present to a healthcare provider with fever. In other words, the patient has returned to his home jurisdiction (where there is no viral outbreak) and develops symptoms associated with dengue fever. In the home jurisdiction, the medical practitioners or health care providers may not know to look for or test for dengue fever as they may not normally check for such a rare disease in the home jurisdictions and/or may not be aware of the patient's travel to any infected area. With a careflow/workflow that incorporates data related to travel to certain geographic areas where there is such an outbreak, it would be easier to properly diagnosis the patient's condition and proceed with the proper treatment. By pulling such relevant information into the careflow and/or the workflow, clinically relevant information may be brought to the attention of the healthcare provider; it does not dictate what the healthcare provider is to do but simply provides the relevant information and at the relevant time (e.g. providing context awareness). In other words, the embodiments of the present invention pull the “right” information at the “right” moment of the careflow, workflow, etc. The information may be provided in the electronic medical record but may be brought to the attention of the healthcare provider at the relevant time (e.g. typically at, before or during the point of treatment, namely “in-process”).

This context awareness may also be relevant to the MOH as it may suggest that the MOH consider whether to modify or change the applicable workflow and/or careflow, as will be described in greater detail below.

The careflow engine executes clinical practice guidelines or workflows as dictated by the applicable entity (e.g. a ministry of health or MOH). It may be possible or desirable to receive careflow configurations and/or updates thereto and incorporate these in to the careflow engine to modify the careflow. The configurations may be received for MOH or other applicable agencies. During the course of careflow and/or workflow execution, the careflow engine of the present invention requests patient information (e.g. an electronic record, such as a SHR) stored in the database. The database responds to these requests by retrieving the desired SHR in whole or in part and then sending the desired information to the careflow engine. At certain points, the workflow may instruct or suggest that an action be taken to provide or suggest a patient with a course of treatment recommended by the MOH. Requests to perform one or more actions in accordance with the clinical practice guideline recommendation may be provided automatically to healthcare workers via their mobile device, other “smart” device, and/or an IoT device. Using the system of the present invention, workflows and the recommendations provided therein can be automatically translated into clinical outcomes without further intervention.

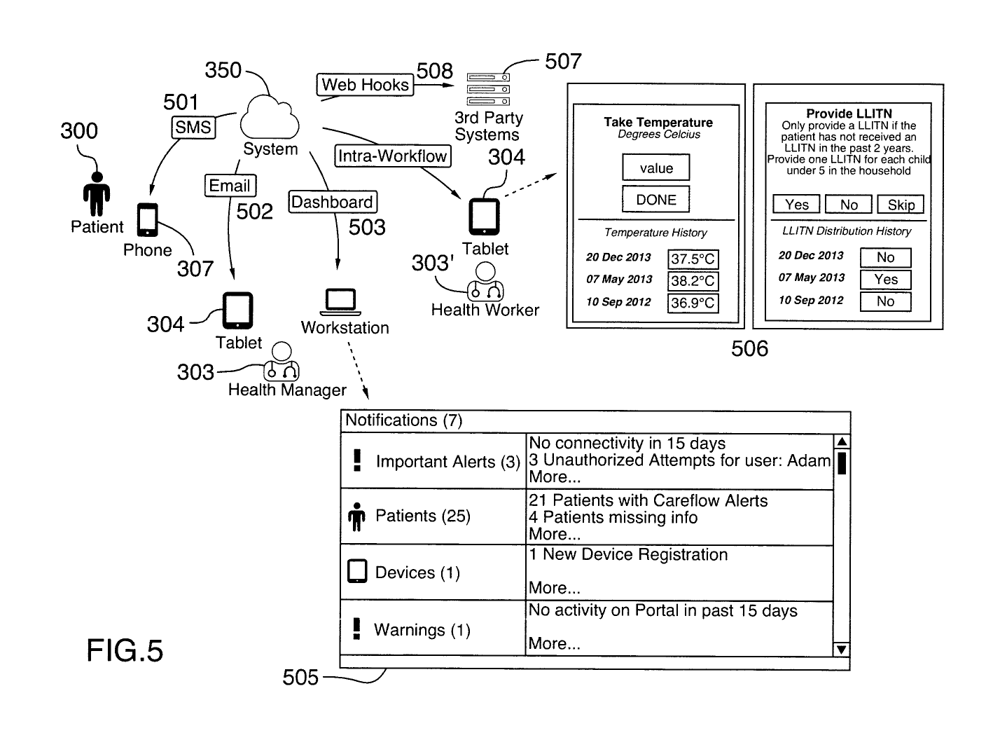

The careflow engine of the present invention executes a careflow, workflow and/or clinical practice guideline and implements an action responsive to execution of the careflow, workflow and/or guideline. During the course of conducting or processing the applicable careflow, workflow, clinical practice guideline, etc., the careflow engine may obtain various types of data as called for by the applicable careflow, workflow, clinical practice guideline, etc. For example, the careflow engine may require various types of patient data in order to process the applicable activity. To gather this information, the careflow engine may interface with various databases or data stores as need (see FIG. 5, for example). The careflow engine may also obtain locally stored patient or non-patient specific data such as time interval data, or other data, in order to carry out process steps in the guideline. A preferred aspect of the present invention is the pulling a diversity of data into the system of the present invention. Typically, previous systems only incorporate clinical data and demographic data related to the patient. With the embodiments of the present invention, clinical data, medical events, demographic data, and other database information typically not included in the health care system, may be combined. Such other database information may not be provided in a simple EMR or part of the overall information related to the patient.

An aspect of the invention also provides that the data provided could be “scored” or have a form of quality control for the data such as that provided in PCT Application No. PCT/CA2012/001066, for example, the contents of which are incorporated herein by reference.

In a preferred embodiment, as shown in FIG. 2, there is shown a high-level model of a careflow in accordance with the present invention and how it related to medical events. Careflows may be structured and conditional, allowing for complex scenarios to be defined. It may be understood that “complex scenarios” are protocols and guidelines which include large heterogeneous data set (clinical demographic, etc.) coming from internal and external sources (e.g. careflows are not simple single branch process steps). The traversal of a patient through a careflow may depend on what medical events have occurred in their history or are expected to occur. As shown in FIG. 2, a careflow consists of one or groups of workflows, each workflow triggered by which would be understood by a person skilled in the relevant art to be referred to as an “encounter” (210). An encounter is typically a type of interaction between the patient and some aspect of the healthcare system. A workflow is a set of steps that the health worker must perform (e.g. asking simple questions, checking basic vitals or running diagnostic tests using specific medical devices or test devices). An application may facilitate the steps necessary to implement a specific workflow. Different technology providers develop different applications to enable one or more parts of, or the entire, workflow.

Where there is a high proliferation of data sources and medical technologies, preferred embodiment of the present invention provide a system may be further supplemented in situations with the following components: (a) data exchange services; (b) interconnectivity services; and (c) relationship, location and travel mapping engine (see FIG. 1).

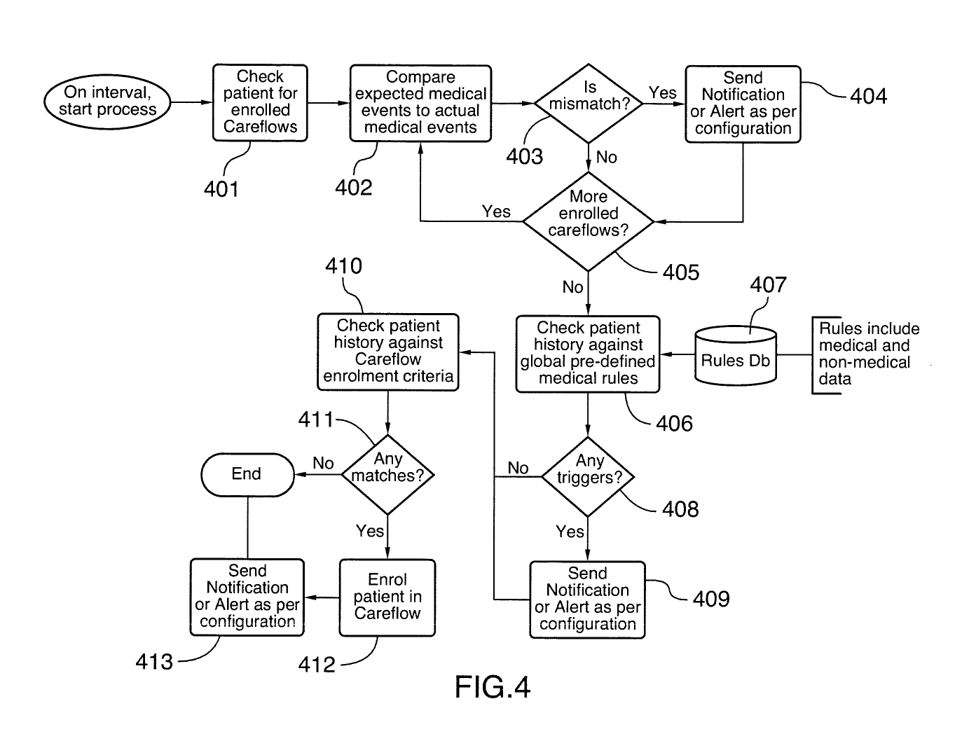

FIG. 4 provides a description of the general operation of the careflow. At 401, the careflow engine may lookup whether the patient 300 (see FIG. 3) is enrolled in an existing careflow(s). Upon confirmation that patient 300 is enrolled in a careflow, the careflow engine may compare the expected medical events to those that have actually occurred (see 402). If there is no inconsistency from what was expected to what was found, then the careflow engine may repeat the process (see 405 in FIG. 4) for all careflows that a patient may be registered. If there are one or more inconsistency, however, then a notification or alert may be sent as per the configuration of the notification component 130.

If there may be no other careflows in which patient 300 may be currently enrolled (406), the careflow engine of the present invention may check the shared health information of patient 300 and compare it against pre-defined medical rules 407 that are applicable as established externally (e.g. by a MOH) and/or based on medical and non-medical data. Should there be a “trigger”, “flag” or a “match” of the medical rules set out 407 as indicated in 408 then a further notification can be sent 409. Where there are no triggers, or once all the triggers have been handled, the careflow engine can then check patient's history against other careflow enrollment criteria to determine if there are other matching criteria 410 and where there are, the patient could be enrolled in other careflow(s) 412 and the applicable notifications can be sent 413.

FIG. 5 sets out preferred embodiments for providing notification or alerts in accordance with the present invention. In FIG. 5, the system may be also shown in use with various devices, preferably including, without limitation, a smart device, an integrated cell phone and reusable test device, an integrated cell phone and consumable test device, and dedicated test devices. It may be understood that the embodiments of the present invention may be used with a number of other devices, including, a desktop computers, cellular telephones, laptop computers, a mobile communications device (e.g., a smart phone), a personal digital assistant, the dedicated test device, and an Internet terminal. The possible devices may preferably also include navigation devices, digital audio players, cameras, gaming devices, televisions, and radios, among others. The preferred devices may preferably be in wireless (and/or wired) communication with one or more of the networks.

As can be seen in FIG. 5, there may be provided difference mechanisms by which the embodiment of the present invention provide notifications or alerts. These include, for example, but are not limited to, SMS (501) to a mobile enabled devices such as mobile phone 307, which may be sent to any type of stakeholder (e.g. anyone who interacts with the system, including, but not limited to, health care providers, MOHs, patients, etc.); email (502) to an internet enabled device such as tablet 304, which may be sent to any type of stakeholder with such a device including health manager 303; and system dashboards (503) of systems or applications being used on any internet connected device, which can include, for example, a dashboard (505) on a web portal viewable on workstation 504 by any stakeholder including health manager 303. Notification dashboards can also be on mobile devices (not shown). Notifications and Alerts can be provided using in-process visual updates, such as those provided in FIG. 5 (see 506). Those provided relevant information, alerts, and/or data in process within a workflow being conducted, for example, in-process, on tablet 304 by health worker 303′. Alerts can be sent to other external systems 507 using web hooks 508 to provide real-time updates to these external systems as necessary.

### Data Store

A preferred embodiment of the present invention provides a system that may have a data storage (e.g. data store 360 in FIG. 3) that may be used to store all necessary data required for the operation of the system. A person skilled in the relevant art may understand that a “data store” refers to a repository for temporarily or persistently storing and managing collections of data which include not just repositories like databases (a series of bytes that may be managed by a database management system (DBMS)), but also simpler store types such as simple files, emails etc. A data store in accordance with the present invention may be one or more databases, co-located or distributed geographically. The data being stored may be in any format that may be applicable to the data itself, but may also be in a format that also encapsulates the data quality.

As shown in FIG. 3, various data stores or databases may interface with the system of the present invention, preferably including, without limitation, proprietary databases, epidemiologic databases, medical records databases, UN and major/international healthcare institution databases, healthcare and emergency infrastructure databases, education and economic databases, news databases, demographic databases, communication and military infrastructure databases, and weather, travel, topographic databases.

A clinical and healthcare database may preferably contain, among other things, diagnostic and medical data (clinical information), such as, for example, one or more of the following, which may or may not be related to medical events: (a) test results from diagnostic devices equipped with remote data transfer systems and/or global positioning or localization features; (b) information from UN databases and major healthcare international institutions; and/or (c) scenarios and knowledge data.

A sociological database may preferably contain, among other things, sociological data (human information), such as, for example, one or more of the following: (a) population information from local and/or international demographic databases; (b) political and/or organization systems in the area and/or from international databases; (c) education and/or economic systems in the area and/or from international databases; and/or (d) information from news and/or newspapers, drawn from the Internet or elsewhere.

An infrastructure database may preferably contain, among other things, infrastructure data or information, such as, for example, one or more of the following: (a) information concerning healthcare infrastructure; (b) information concerning communication infrastructures; and/or (c) information concerning emergency and/or military infrastructure; all preferably drawn from local and/or international databases.

A geophysics database may preferably contain, among other things, geophysics data or information, such as, for example, one or more of the following: (a) weather and/or climatic information from local databases; and/or (b) topographic information from local and/or international databases.

### Shared Health Record System

A preferred embodiment of the present invention provides a system that may have a shared health record (“SHR”) which may be comprised of the patient's medical and non-medical history, including medical events or MEs, as well as associated medical and non-medical data. In a preferred embodiment, the SHR may be provided in a data store. A person skilled in the relevant art may understand that a patient medical and non-medical history or record, preferably in electronic form, may comprise an amalgamation of relevant medical and non-medical information that may be related to the patient. A person skilled in the relevant art may further understand that the patient's medical and non-medical history as well as associated medical and non-medical data may be provided in an electronic medical record (“EMR”), which may be an electronic or digital version of the paper-based medical record for an individual. A person skilled in the relevant art may understand that an EMR contains the standard medical and clinical data gathered in one location (e.g. a single health care provider's office). A person skilled in the relevant art may understand that an electronic health record (“EHR”) goes beyond the data collected in a single location and may include a more comprehensive patient history. For example, EHRs are designed to contain and share information from all providers involved in a patient's care. EHR data can be created, managed, and consulted by authorized providers and staff from across more than one health care organization. It may be understood by a person skilled in the relevant art that the terms EMR and EHR may be used interchangeably in the present application. It may be further understood that the SHR, in a preferred embodiment, contains more information than the EHR or the EMR. In a preferred embodiment, examples of the information that may be included in, but are not limited to, an SHR comprise information related to: (a) patient visit(s) to a facility; (b) patient blood pressure measurement; (c) patient diagnosed with malaria; (d) patient did a regular follow-up; (e) patient was administered specific medicine; (f) patient's recent travel history; and/or (g) patient outcome subsequent to treatment.

Preferred embodiments of the present invention may contain a shared health record system that may obtain and store all applicable patient medical and non-medical information in the data store. Medical and/or non-medical information can be stored with all the relevant associated data including but not limited to: (a) healthcare or health worker(s) who part of an event, if any (for example, Dr. Smith conducted a follow-up examination); (b) facility at which the event took place, if any (for example, follow-up examination was conducted at St. Michael's Hospital); (c) condition for which the event was conducted, if any (for example, follow-up was scheduled due to Malaria diagnosis earlier in the year); (d) medical service for which the event was conducted, if any (for example, a follow-up that may be part of a community outreach program for essential health services). This allows the system to dissect the information across multiple dimensions as necessary. An example of this could be the query for all health events associated with a specific condition across a particular healthcare level in the system.

### Established Protocols for the Workflows

A person skilled in the relevant art may understand that health care protocols or workflows refer to medical guidelines, clinical guideline, clinical protocol or clinical practice guidelines, that guiding decisions and criteria regarding diagnosis, management, and treatment in specific areas of healthcare. Such healthcare protocols are typically established by applicable governmental (e.g. applicable Ministries of Health (“MOH”)) or non-governmental agencies (e.g. Canadian Medical Association, World Health Organization, etc.) for their respective regions or nations. These healthcare protocols may be vary by region or may be based on widely accepted protocols which are sponsored by popular public health agencies, such as the World Health Organization. These protocols are usually a set of procedure or actions that must be performed to deliver quality care.

The system of the present invention, in a preferred embodiment, may consider these protocols and their procedures, actions, and outcomes as expected patient medical events. They are no different than actual patient medical events with the exception in that they have not occurred yet but are expected to occur in the future, and in some cases at a specific time. The system may provide a means for experts, such as personnel of Ministries of Health, to enter and manage pre-defined protocols. The system may not help define healthcare protocols, as that may be the purpose of experts at the Ministry of Health. The system may provide the means to configure those protocols into a language that the system of the present invention can understand. Furthermore, the system may allow the management of those protocols, allowing for a control mechanism to support governance of protocols by Ministries of Health. In a preferred embodiment, these configure protocols may be included within a careflow.

### Medical Events and Careflow Definition

A preferred embodiment of the present invention defines a structured model for medical events, careflows, workflows, etc. FIG. 2 showcases the high-level model of the embodiments of the present invention and how it relates to medical events. As shown in FIG. 2, there may be provided a preferred embodiment in which the careflow comprises of one or groups of workflows, defined by the patient encounters.

### Careflow Engine

The careflow engine component of the system may be responsible for monitoring actual patient medical events and comparing them to actual and/or expected patient medical events. The shared health record component stores all patient medical events, whether actual or expected. The careflow definition defines the expected future medical events.

The careflow engine may be context aware and may proactively: (a) evaluate actual and expected patient medical events; (b) determine the next logical medical event and the timing for that event; (c) determine if any deviations between the actual events or missing events (from the shared medical record) and expected events (from the careflow); (d) determine if there are relevant historical patient records related to the current interaction, or to a specific portion of the current interaction (see in-process alerts in the notifications engine section); (e) determine if there are disease risk factors and automatically enroll patients into additional careflows (such factors are identified when the system links the current patient to other patients that may imply health risks. For example, a link by co-habitation with patients known by the system to be currently positive for a highly infectious disease such as Tuberculosis; a familial link to another patient, perhaps a brother, known to have a severe hereditary disease. Other links may be established using geo-temporal associations, flight numbers and passenger manifests); and/or (f) notify appropriate stakeholders based on system settings (see notifications engine section).

Notification and/or Alerts Engine

The notification and/or alerts engine component of the system may be responsible for distributing notifications as configured. The notification engine may support distribution via numerous means including, but not limited to, In-process during protocol execution, Email, SMS, Web Hooks and System Dashboards and other methods currently known or developed subsequently.

The notification engine may be configurable to allow notifications based on: (a) any alert on a careflow, including missed medical events, extra unexpected medical events, improper timing of medical events or incorrect sequencing of medical events. For example, if a patient should have had a follow-up visit for their HIV treatment but it was missed, the system would alert the patient and the healthcare worker via SMS; (b) alert on certain event types not limited by careflow, such as notification of all diagnostic test results. For example, a lab manager could configure the system to email them whenever a new positive diagnostic test may be done; (c) alert on enrollment of patient in careflow, such as diagnosing a patient with a disease and putting them on the appropriate protocol. For example, when a patient may be diagnosed with Ebola and enrolled into the Ebola careflow, an email could be sent to a national registry that performing disease surveillance; (d) alert in-process during protocol execution to provide additional context information to health worker during care delivery. For example, if a patient has a history of high blood pressure at different clinics, the notification engine would make the protocol engine (and thereby the health worker) aware of this information at the moment a blood pressure measurement may be being taken. This would allow the health worker to consider the aggregate information during their evaluation; (e) alert health workers or patients that a medical event has been missed—For example a missed appointment for antenatal care could be flagged and an email sent to the health worker and a SMS sent to the patient with a reminder, phone number and potential appointment time; (f) Alert health workers that a patient has been lost during referral. For example, a patient referred from a clinic to a hospital for a speciality appointment does not follow up and the system can notify both the health workers and patient about the missed appointment and what it means in the treatment of the particular medical condition

### Data Exchange and Interconnectivity Components

It's clear from the description of the system that the system's overall usefulness and value stems from its ability to acquire all necessary patient information, including medical events. As such, in situations where the system may be implemented in an environment where there may be a high proliferation of data sources and medical technologies, it should be supplemented with two extra components.

The data exchange component may be composed of a set of services that support the automated import/export of data with numerous 3rd party data platforms. While the data query and transfer are not novel, the data exchange component may provide a means to establish uniqueness of a patient via the use of a patient identification service. This patient identification service may serve as a repository of patients and all their associated identities across different systems. Once the relationship between the system's patient identity may be matched with the identity of patient from 3rd party data platforms, the data can be automatically queried and added to the patient's shared health record.

The interconnectivity component may be composed of a set of services that provide the capability to 3rd party technology systems to directly add and query the patient's shared health record. This allows 3rd party technology systems to leverage the shared health record and patient identification capabilities of the system while adding to the patient's shared health record ensuing it has a complete history of the patient's medical events. In an embodiment of the present invention, examples include an API to allow 3rd party systems query for a patient's previous blood pressure results, and query for a patient's family history related to hypertension. The same 3rd party system could then use an API to submit their own readings related to hypertension/BP measurements. An API to allow 3rd party systems to do a lookup of a patient using their demographics (i.e. first name, last name, dob and last visit to facility). This lookup would allow them to further lookups into patient history and patient specific alerts (such as missed appointments, etc). Basically there may be Service APIs to support every claim in this patent submission including, but no limited to: (a) obtaining patient demographics; (b) patient look-ups using demographics; (c) obtaining patient historical medical events (EHR); (d) obtaining outstanding patient alerts; (e) obtaining patients non-medical event history (travel, etc.); (f) obtaining patient's expected future medical events; (g) providing an alert to the system to be used by other system component, health care providers or other users (for example—if the 3rd party system happens to be a system for monitoring a new infectious disease, the system could provide an alert to the careflow system thereby alerting all health managers of facilities that a patient has recently been in contact with to control disease transmission).

### Relationship, Location and Travel Mapping Component

As part of collecting patient demographics, relationship maps can be established between patients allowing for family trees, spousal details, co-worker relationship along with various other relationship models. Much of this information may be collected during regular patient visits, but are also collected during other types of patient encounters, for example during infectious disease response in the form of contact tracing. Other related information that may be collected and mapped may be also considered, for example, locations visit in the past, travel information including flights taken and geo-location of social media updates (i.e. twitter updates from Malaysia). All of this information may be always time-stamped allowing the creation of network maps that may correlate one or more of the following: (a) Family network map (i.e. father-child, husband-wife, etc.); (b) Relationship network map (i.e. coworkers, friends, etc.); (c) Contact location map (i.e. met with John at Bar, met with Cindy at park); (d) Location History (i.e. was in downtown Toronto yesterday at 2 pm, was in Kenya last week);and/or (e) Travel details (i.e. travelled on flight AC2233 from Toronto to Cape Town on Jul. 22, 2015).

This type of information can help with disease tracking, such as Zika and Ebola, by allowing the system to correlate location history with known hotspots and alerting the health worker in-process during protocol execution. Disease specific factors can be tuned in the protocols and used for tracking—for example Zika virus may be spread by mosquito bites in urban indoor settings during the day time, Ebola may be spread through bodily contact. Both of these examples can highlight specific questions to ask the patient in process. It can also add additional context to symptoms, for example by showcasing travel history while taking temperature and blood pressure readings.

While the description of the invention disclosed herein may be provided in the health care context, the invention itself may be a system guides a user through an interaction with a target (the patient in the above) on the basis of historical information, protocols, and expected outcomes. The invention could be applied to interactions in other sectors including but not limited to encounters at national ports of entry, encounters with law enforcement officials, retail encounters between a vendor or waiter and a patron, the food inspection industry, and bio-threat applications.

There are general subsystems that allow for the sharing of information, configuration, administration and modification of the information within the system of the presentation invention. There are existing systems that allow for exchange of information between devices.

The foregoing description has been presented for the purpose of illustration and may be not intended to be exhaustive or to limit the invention to the precise form disclosed. Other modifications, variations and alterations are possible in light of the above teaching and may be apparent to those skilled in the art, and may be used in the design and manufacture of other embodiments according to the present invention without departing from the spirit and scope of the invention. It may be intended the scope of the invention be limited not by this description but only by the claims forming a part of this application and/or any patent issuing herefrom.

1-45. (canceled)

46. A system for context-awareness for in-process patient care management comprising:
(a) a first database in communication with a network and configured to store therein a shared patient record of a patient and at least one patient careflow for treatment of a condition of the patient;
(b) a careflow engine in communication with the network and configured to receive the shared patient record and the at least one patient careflow therefrom and to analyze the careflow in the context of the shared patient record in order to create a clinical careflow;
(c) a notification engine in communication with the network and configured to receive the clinical careflow from the careflow engine and to communicate the clinical careflow to a health care provider who is co-located with the patient during or before the time of treatment of the patient;
(d) a network ready device in communication with the network and configured to display the clinical careflow to the health care provider through a health care provider interface for the health care provider to guide the treatment of the condition of the patient with the clinical careflow;
wherein the patient careflow further comprises treatment workflows;
wherein the shared patient record further comprises medical and non-medical patient specific information;
wherein the medical and non-medical patient specific information is selected from the group consisting of healthcare providers having encounters with the patient, internal patient factors and external patient factors; and
wherein the external patient factors comprise geographic information, social media information, non-medical personal information, and/or mixtures thereof.

47. The system of claim 46, wherein the shared patient record comprises an electronic health record.

48. The system of claim 47 wherein the electronic health record further comprises an electronic medical record.

49. The system of claim 46 wherein the internal patient factors comprise the patient's medical history.

50. The system of claim 46, wherein the treatment workflows comprise clinical practice protocols or guidelines.

51. The system of claim 50 wherein the careflow engine evaluates the patient careflow and the shared patient record to determine the next treatment workflow in the treatment of the condition of the patient.

52. The system of claim 51 wherein the careflow engine evaluates the patient careflow and the shared patient record to determine if deviations from the patient careflow have occurred.

53. The system of claim 52, wherein the careflow engine is configured to receive data from a second database, where the second database is selected from a group consisting of proprietary databases, epidemiologic databases, medical records databases, UN and major/international healthcare institution databases, healthcare and emergency infrastructure databases, education and economic databases, news databases, demographic databases, communication and military infrastructure databases, and weather, travel, topographic databases.

54. The system of claim 52 wherein the notification engine is configure to anonymize the clinical careflow and to communicate the anonymized clinical careflow to a governmental or non-governmental agency responsible for the patient careflow.

55. The system of claim 54 wherein the healthcare provider is selected from the group consisting of a primary care physician, a nurse practitioner, a clinic, a hospital, a physician's assistant, a therapist, a specialist, an insurance carrier, a healthcare payer, a pharmacist, an accountable care organization, and a hospice provider.

56. The system of claim 55 wherein the first database, the second database, the careflow engine, the notification engine and the network ready device are configured to communicate securely using at least one of email, short message service (SMS), secure mobile messaging, web hooks, system dashboards, interactive voice response.

57. A method for providing context-awareness during in-process patient care comprising:
(a) storing an electronic record of a patient and at least one patient careflow for treatment of a condition of the patient in a first database communicating with a network;
(b) transmitting the electronic record of a patient and the at least one patient careflow through the network to a careflow engine communicating with the network, the careflow engine analyzing the careflow in the context of the electronic record of a patient and creating a clinical careflow;
(c) transmitting the clinical careflow through the network to a notification engine communicating with the network, the notification engine transmitting the clinical careflow to a network ready device of a health care provider who is in-process with the patient during or before the time of treatment of the patient;
(d) displaying the clinical careflow to the health care provider through a health care provider interface in the network ready device of the health care provider guiding the treatment of the condition of the patient;
wherein the patient careflow further comprises treatment workflows;
wherein the electronic health record further comprises medical and non-medical patient specific information;
wherein the medical and non-medical patient specific information is selected from the group consisting of healthcare providers having encounters with the patient, internal patient factors and external patient factors; and
wherein the external patient factors comprise geographic information, social media information, non-medical personal information, and/or mixtures thereof.

58. The method of claim 57, wherein the electronic record of a patient comprises an electronic health record.

59. The method of claim 58, wherein the electronic health record further comprises an electronic medical record.

60. The method of claim 57, wherein the internal patient factors comprise the patient's medical history.

61. The method of claim 57, wherein the treatment workflows comprise workflows comprise clinical practice protocols or guidelines.

62. The method of claim 61 wherein the careflow engine evaluates the patient careflow and the shared patient record to determine the next treatment workflow in the treatment of the condition of the patient.

63. The method of claim 62 wherein the careflow engine evaluates the patient careflow and the electronic record to determine if deviations from the patient careflow have occurred.

64. The method of claim 62, wherein the careflow engine is configured to receive data from a second database, where the second database is selected from a group consisting of where the second database is selected from a group consisting of proprietary databases, epidemiologic databases, medical records databases, UN and major/international healthcare institution databases, healthcare and emergency infrastructure databases, education and economic databases, news databases, demographic databases, communication and military infrastructure databases, and weather, travel, topographic databases.

65. The method of claim 62 wherein the notification engine is configure to anonymize the clinical careflow and to communicate the anonymized clinical careflow to a governmental or non-governmental agency responsible for the patient careflow.

66. The method of claim 65 wherein the healthcare provider is selected from the group consisting of a primary care physician, a nurse practitioner, a clinic, a hospital, a physician's assistant, a therapist, a specialist, an insurance carrier, a healthcare payer, a pharmacist, an accountable care organization, and a hospice provider.

67. The method of claim 66 wherein the first database, the second database, the careflow engine, the notification engine and the network ready device are configured to communicate securely using at least one of email, short message service (SMS), secure mobile messaging, web hooks, system dashboards, interactive voice response.

68. A non-transitory computer readable medium encoded with executable instructions for providing context-awareness during in-process patient care, the executable instructions comprising code for:
(a) storing an electronic record of a patient and at least one patient careflow for treatment of a condition of the patient in a first database communicating with a network;
(b) transmitting the electronic record of a patient and the at least one patient careflow through the network to a careflow engine communicating with the network, the careflow engine analyzing the careflow in the context of the electronic record of a patient and creating a clinical careflow;
(c) transmitting the clinical careflow through the network to a notification engine communicating with the network, the notification engine transmitting the clinical careflow to a network ready device of a health care provider who is in-process with the patient during or before the time of treatment of the patient;
(d) displaying the clinical careflow to the health care provider through a health care provider interface in the network ready device of the health care provider guiding the treatment of the condition of the patient;
wherein the patient careflow further comprises treatment workflows;
wherein the electronic health record further comprises medical and non-medical patient specific information;
wherein the medical and non-medical patient specific information is selected from the group consisting of healthcare providers having encounters with the patient, internal patient factors and external patient factors; and
wherein the external patient factors comprise geographic information, social media information, non-medical personal information, and mixtures thereof.

69. The computer readable medium of claim 68, wherein the electronic record of a patient comprises an electronic health record.

70. The computer readable medium of claim 69 wherein the electronic health record further comprises an electronic medical record.

71. The computer readable medium of claim 68 wherein the internal patient factors comprise the patient's medical history.

72. The computer readable medium of claim 68, wherein the treatment workflows comprise clinical practice protocols or guidelines.

73. The computer readable medium of claim 72 wherein the careflow engine evaluates the patient careflow and the shared patient record to determine the next treatment workflow in the treatment of the condition of the patient.

74. The method of claim 73 wherein the careflow engine evaluates the patient careflow and the electronic record to determine if deviations from the patient careflow have occurred.

75. The computer readable medium of claim 73, wherein the careflow engine is configured to receive data from a second database, where the second database is selected from a group consisting of where the second database is selected from a group consisting of proprietary databases, epidemiologic databases, medical records databases, UN and major/international healthcare institution databases, healthcare and emergency infrastructure databases, education and economic databases, news databases, demographic databases, communication and military infrastructure databases, and weather, travel, topographic databases.

76. The computer readable medium of claim 73 wherein the notification engine is configure to anonymize the clinical careflow and to communicate the anonymized clinical careflow to a governmental or non-governmental agency responsible for the patient careflow.

77. The computer readable medium of claim 76 wherein the healthcare provider is selected from the group consisting of a primary care physician, a nurse practitioner, a clinic, a hospital, a physician's assistant, a therapist, a specialist, an insurance carrier, a healthcare payer, a pharmacist, an accountable care organization, and a hospice provider.

78. The computer readable medium of claim 77 wherein the first database, the second database, the careflow engine, the notification engine and the network ready device are configured to communicate securely using at least one of email, short message service (SMS), secure mobile messaging, web hooks, system dashboards, interactive voice response.
